(19) United States
(12) Patent Application Publication
(10) Pub. No.: US 2019/0105040 A1
(43) Pub. Date: Apr. 11, 2019

(54) BONE TIES AND STAPLES FOR USE IN ORTHOPAEDIC SURGERY

(71) Applicant: APIO IMPLANTS LIMITED, Harrow Middlesex (GB)

(72) Inventor: David Gordon, Harrow Middlesex (GB)

(73) Assignee: APIO IMPLANTS LIMITED, Harrow Middlesex (GB)

(21) Appl. No.: 16/087,219

(22) PCT Filed: Mar. 24, 2017
(86) PCT No.: PCT/GB2017/050836

§ 371 Date: Sep. 21, 2018

(30) Foreign Application Priority Data
Mar. 24, 2016 (GB) 1605087.4

(51) Int. Cl. A61B 17/064; A61B 17/80; A61B 17/70

(52) U.S. Cl. CPC A61B 17/0642; A61B 17/8009; A61B 17/8019; A61B 17/8023; A61B 17/7059

(57) ABSTRACT

The present invention concerns the field of orthopaedic surgery, veterinary surgery, and maxillofacial surgery. The invention may find application in other fields such as dentistry or where joining, fusing or stabilising of two bones (or one bone and one prosthetic member) are required. There invention provides a surgical bone tie for use in joining abutting bone surfaces for fusing or knitting of the bone together, the tie comprising (or consisting of) first and second pieces each having a proximal end region and a distal end region which define a longitudinal axis therebetween, wherein the distal end regions are each provided with a mounting feature for fixing, or permitting the fixing, of each piece to respective underlying portions of bone, wherein the proximal end regions of each pieces are adapted for engagement together along said axis so that one proximal end region may be accommodated by the other proximal end region to provide a bridge between the respective distal end regions, the engagement permitting one way travel of one piece progressively towards the other so that the bridge length becomes progressively smaller until a desired amount of separation between the distal end regions is obtained, and the a desired compression between the abutting bones is obtained. The engagement between the proximal end regions preferably comprises a ratchet and pawl connection.

### CROSS REFERENCE TO RELATED APPLICATION

The present application is the U.S. national stage application of International Application PCT/GB2017/050836 filed Mar. 24, 2017, which international application was published on Sep. 28, 2017 as International Publication WO 2017/163080 A1. The International Application claims priority to Great Britain Patent Application 1605087.4 filed Mar. 24, 2016.

### FIELD OF THE INVENTION

The present invention concerns the field of orthopaedic surgery, veterinary surgery, and maxillofacial surgery. The invention may find application in other fields such as dentistry or where joining, fusing or stabilising of two bones (or one bone and one prosthetic member) are required.

### BACKGROUND OF THE INVENTION

In orthopaedic surgery, a method of treating a worn and painful joint is to fuse the two facing joint surfaces together. Fusion is where two raw bone surfaces come into contact with each other and biologically join together by the growth of bridging bone. A biological fusion requires compression and stability. Integration or stabilization of prosthetic implants may also be required in patients. Current devices for these purposes used to facilitate fusion include screws, plates (with screws) and bone staples.

Screw fixation is commonly used but often requires multiple screws to achieve stability, drilling to create a pilot hole which may cause bone necrosis (cell death) and disruption of the blood supply which is crucial for fusion healing.

Bone staples have advantages over other implants due to their ease of insertion which therefore reduces operation time involved for implanting. As shown in FIG. 1A surgical bone staples 1 are each typically comprised of two limbs 2, 3 and a bridge 4 which joins them. Current staple designs include static staples that do not allow for compression, and compression staples.

Compression staples provide compressive forces which aid biological fusion by urging facing bone surface together. They compress either by a manipulating the structure (such as by using a tool) or by the use of memory metals such as Nitinol (RTM). For example, in the staple 5 shown in FIG. 1B, a tool (not shown) may be used to spread apart two side-by-side bridging rods 6 which connect the two staple limbs (provided by screws 7). This shortens the distance between the two limbs 7. The unspread configuration is shown in dotted lines in the figure. A disadvantage of this design is that compression forces obtained are weak, and the forces may reduce overtime due to material relaxation. Furthermore staples may cause distraction at the opposite side of bone, causing a gap and potential non-union of the fusion site (see Farr D et al, “A biomechanical comparison of shape memory compression staples and mechanical compression staples: compression or distraction?” Knee Surg. Sports Traumatol. Arthrosc. 2010;18:212-7 and Shibuya N et al, “A compression force comparison study among three staple fixation systems.” J. Foot Ankle Surg. 2007; 46:7-15.)

Memory metals are materials which can return to a predefined shape, so for a staple, following insertion in the body, the limbs return to a compressive state, where the distance between the limb tips (2, 3 in FIG. 1C) are biased to reduce. With some designs of current staples (due to a hinge effect where the limbs join the bridge) compression forces reduce along the length of the limb toward the bridge; being greatest at the tip and smallest near the bridge. In addition, the user is unable to select the degree of compression as it is predetermined during the design and manufacture of the staple. Some memory metals require activation using bespoke devices or machines, which increases cost and surgical time.

U.S. Pat. No. 3,604,414 (1971) discloses a two piece osteo-synthesis plate which includes a toothed-rack system to enable the two pieces to be drawn together and locked in place.

FR-A-1,239,266 (1959) discloses a splint for effecting osteo-synthesis of fractures to long limbs. The elongate two-part device has a spring-loaded mechanism for urging the two parts together and a ratchet rack providing one-way axial compressive movement.

U.S. Pat. No. 3,807,394 (1974) discloses a surgical staple in which the bridge between the two staple limbs is formed as a tube in which is disposed a helical spring which is attached at opposite ends thereof to return portions of the limbs which are accommodated in the each tube end. In this way a compressive force may be maintained drawing the two limbs together. There is however no locking mechanism to prevent separation of the limbs under axial loads.

U.S. Pat. No. 5,053,038 discloses a unitary bone staple in which the limbs of the staple are each formed with a splayed lower region and a radiused, resiliently flexible upper region. As the staple is driven into bone, to bridge a joint, the two joined bone regions are compressed together.

U.S. Pat. No. 4,852,558 discloses a bone staple formed in three pieces. The first piece has an L-form with a depending staple limb and a perpendicular bridge portion. The bridge portion has an underside provided with a ratchet rack. The second piece has a depending limp and a perpendicular sleeve portion which slidably receives the bridge portion of the first piece. The third piece is a locking member which has an L-form and has two locking teeth at an end region which is slotted into the second piece sleeve to engage with the ratchet rack of the bridge portion of the first piece. This locks the staple at a desired bridge spacing. The use of a locking member means that when implanting the staple, a two part process is necessary, in which the staple is compressed (by the use of pliers) to the desired bridge length (limb spacing), and then compression is maintained while the locking member is driven home in a separate step. Inevitably there is a tendency for the compression to be released somewhat while the locking member is driven home, which makes the staple less effective.

The present invention seeks to provide an improved surgical bone staple or tie which is capable of maintaining a significant compressive load, but which is simple to implant and which does not tend to relax over time. The compressive strain is selectable according to requirements and to achieve a desired compression.

### SUMMARY OF THE INVENTION

According to one aspect of the present invention there is provided a surgical bone tie for use in joining abutting bone surfaces for stabilizing, fusing or knitting of the bone together, the tie comprising (or consisting of) first and second pieces each having a proximal end region and a distal end region which define a longitudinal axis therebetween, wherein the distal end regions are each provided with a mounting feature for fixing, or permitting the fixing, of each piece to respective underlying portions of bone, wherein the proximal end regions of each pieces are adapted for engagement together along said axis so that one proximal end region may be accommodated by the other proximal end region to provide a bridge between the respective distal end regions, the engagement permitting one way travel of one piece progressively towards the other so that the bridge length becomes progressively smaller until a desired amount of separation between the distal end regions is obtained, and then a desired compression between the abutting bones is obtained.

In a preferred aspect of the invention the engagement between the proximal end regions comprises a ratchet and pawl connection. The ratchet may be provided on or in one proximal end region and the pawl may be provided in or on the other proximal end region.

The first piece of the bone tie is preferably an elongate female sleeve member with a hollow cross-section and the second piece of the bone tie is preferably a male member that forms a sliding fit in the female sleeve member. By using an elongate female sleeve member with hollow cross-section, the elongate female sleeve member fully envelops the proximal end region of the male member in use. The female sleeve member therefore shields the ratchet and pawl connection from impingement by soft tissue at the implantation site.

The pawl preferably comprises a cantilever spring member, a free end of which spring member is biased against the ratchet for sequential engagement with ratchet teeth. By using a cantilever spring the number of moving parts and components is minimized. Use of a cantilever spring can also avoid damage to the pawl or ratchet teeth during compression.

The pawl is preferably a cantilever spring member oriented along an axial direction so as to co-operate with the rack when engaged.

One proximal end region of a tie piece preferably has the form of an elongate sleeve and the other proximal end region has the form of an elongate projection. The projection is preferably a sliding fit in the elongate sleeve.

One or both distal end regions may be provided with an eyelet for receiving a bone screw or bolt. Preferably both distal end regions are provided with an eyelet for receiving a fixing element such as a bone screw or bolt or spike.

One or both distal end regions may be provided with a (possibly generally perpendicularly-oriented) limb to be driven into the underlying bone for fixing thereto. Preferably both distal end regions are provided with a limb to be driven into the underlying bone for fixing thereto.

In a such staple configurations the limbs will be generally perpendicularly oriented, but may have another angles depending upon the form and orientation of the bones to be joined.

The ratchet preferably comprises an axially oriented ratchet rack, each click of which corresponds to a discrete amount of end region separation.

In some embodiments the cantilever spring member projects axially towards the proximal end region of the associated tie piece. In some other embodiments the cantilever spring member projects axially towards the distal end region of the associated tie piece.

The pawl is preferably provided by one piece of the tie and the ratchet is provided by the other tie piece. The pawl and ratchet are preferably integrally formed with each respective tie piece.

The ratchet may be provided along a surface of the projection with ratchet ramps oriented towards the sleeve and the pawl is provided in or on an inner wall of the sleeve as a member which is biased against and into the path of the ratchet, so that the elongate projection ratchets into the sleeve during engagement.

In yet another aspect of the invention there is provided a method of joining two abutting bone portions comprising:

providing at least one surgical bone tie as hereinbefore described,

selecting a location for the tie member to span the bone joint,

fixing each distal end region of the tie to a respective underlying bone portion,

compressing the bone tie in a longitudinal direction so as to achieve a desired separation between the tie distal end regions, and

Optionally applying further surgical bone ties at further locations spanning the bone joint.

The method may also include the step, before compressing, of drilling pilot holes which correspond to an initial bridging length of the bone tie, locating the device so as to be aligned over said pilot holes and fixing the device by applying limbs, screws or bolts to said pilot holes to fix the bone tie device.

### BRIEF DESCRIPTION OF THE DRAWINGS

Following is a description by way of example only and with reference to the figures of the drawings of ways of putting the invention into effect.

In the drawings:—

FIGS. 1A to 1C show various prior art bone staple configurations.

FIG. 2 is a perspective view of a bone tie in accordance with one embodiment of the invention.

FIG. 3 is perspective view in cross-section of the embodiment shown in FIG. 2.

FIG. 4 is a perspective view in cross section of a bone tie which represents an alternative embodiment of the invention.

FIGS. 5A and 5B are schematic plan views of a bone tie in accordance with the invention.

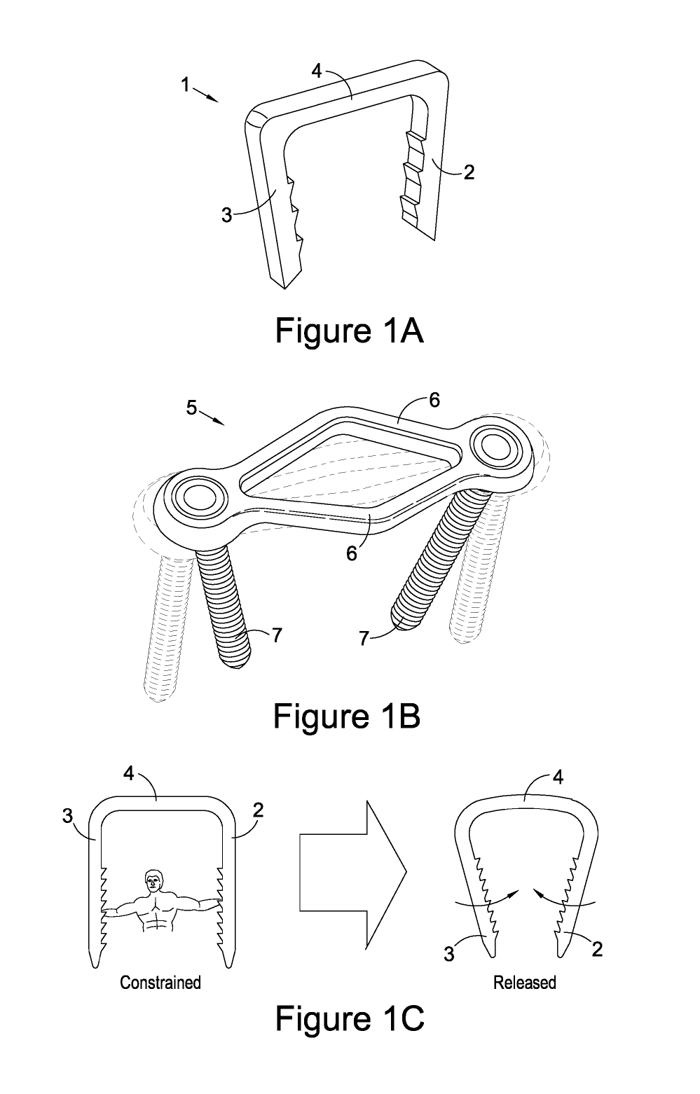

### DETAILED DESCRIPTION

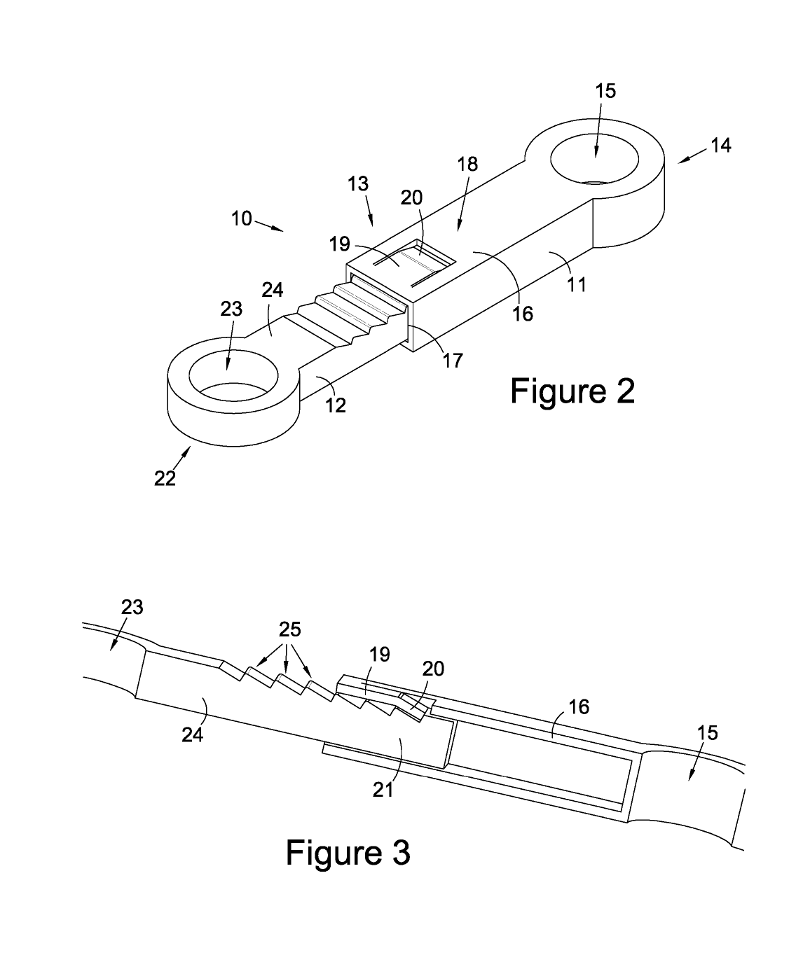

In FIG. 2 a bone tie 10 is shown. The bone tie consists of a first (female) sleeve member 11 engaged with a second (male) member 12. The sleeve member is elongate and has a proximal end region 13 and a distal end region 14. The distal end region is formed with an annular, vertically opening eyelet 15. A body portion 16 of the sleeve member is elongate with a hollow cross section. In this embodiment the body portion 16 of the sleeve member has a rectangular hollow cross section, however it may have alternative cross sectional shapes, such as a rounded-rectangular hollow cross section. The proximal end 17 of the sleeve member is open to permit entry of the male member. An upper surface 18 of the proximal end is formed with a U-shaped rectilinear cut which defines a tongue 19. The tongue 19 has a distal end region which is formed with an elbow which causes the end 20 to depend from the plane of the tongue. The depending end 20 thus serves as a cantilever sprung pawl (see FIG. 3).

The male member 12 is elongate and has a proximal end region 21 and a distal end region 22. The distal end region is formed with an annular vertically opening eyelet 23. A body portion 24 of the male member is rectilinear in cross section and sized to be a sliding fit in the body portion 16 of the sleeve member. Alternatively the body portion 24 of the male member may have other cross sectional shapes, the male member being shaped and sized for a sliding fit in the body portion 16 of the sleeve member. An upper surface of the proximal end region of the male member is formed with ratchet teeth 25 (six teeth shown in FIG. 3). The teeth span the width of the body portion and have a factory roof (or saw tooth) cross-section—which is to say there is a long shallow side and a short steep side to each tooth. The short side serves as a stop against which the pawl 20 can lock. The slope angles of the tooth short side and the pawl abutment surface (end surface) are matched. In other embodiments, the teeth may not span the width of the body portion, but span at least a portion of the width, such as a mid-portion of the width of the body portion.

As is shown in FIG. 3 as the male member progresses into the sleeve member, the pawl is resiliently displaced as it passes each ratchet tooth. In the embodiment shown in FIG. 3 there are seven possible pawl lock positions in which the pawl is seated against a (tooth short side) abutment. The pawl produces an audible click as each tooth is passed.

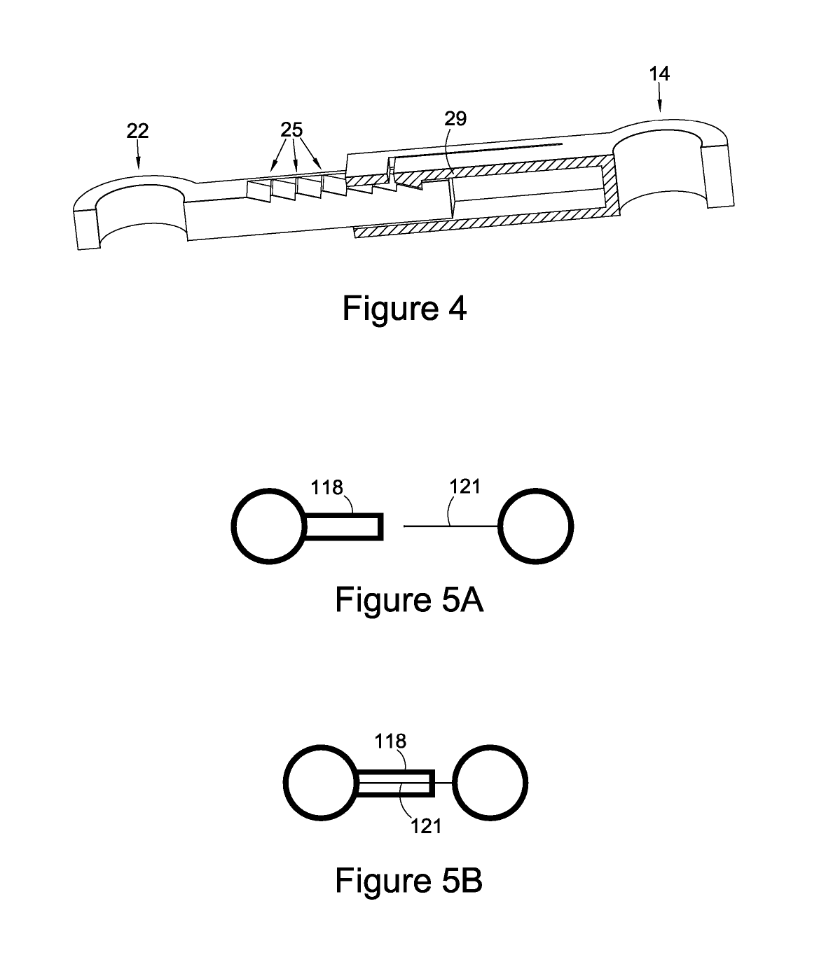

FIG. 4 shows an alternative embodiment in which the pawl 29 is provided by a tongue cut out in the sleeve which projects towards the sleeve proximal end 30 rather than towards the distal end 31. The free end of the tongue is provided with a non-return barb which engages with the ratchet teeth 25.

Alternative arrangements may also be envisaged in which the ratchet is provided along a sleeve internal surface (rather than on a male member surface) and the pawl is provided on the male member, such as by (one or more) cantilever tongues provided on the male member outside surface.

In place of eyelets there may be limbs which are adapted for fixing in bone. So they may be depending (in the manner of a staple), they may have sharp ends and the may have barbs to help prevent unwanted removal.

The orientation of the limbs may vary according to a range of pre-selected combinations, or may be tailored by the surgeon or technician at the time of entry by bending. In a basic embodiment the limbs are oriented perpendicularly downwards to as to facilitate fixing to underlying bone surfaces. Any angle may be employed to allow anatomical conformity to the area being fused. E.g. a 20 degree angle to accommodate the curve in a mid-foot fusion.

Each limb maybe formed as a flat spade-like part (flat, thin, possibly curved) rather than a spike. This means that compressive forces may be distributed over a larger area. This may prevent ‘cheese cutting’ of the limb during the application of high compressive forces, or in response to patient mobility. Each of the staple dimensions may be varied—the limb lengths (which may be different on the same staple), the bridging portion length (pre compression) and any of the components' widths. This may allow an increase in rigidity and use in a range of anatomical locations, both small and large bones.

In summary the engagement mechanism may be an internal ratchet, comprising of a male and female (sleeve) members. The female member may have a pawl on its top surface or one or two pawls on its side surface. The pawl may be a cantilever spring which has its base (attached portion) directed toward the proximal end or the other distal end.

Typically the axial pawl may vary in length from 2 or 3 mm or be even longer (greater than 3 mm). The pawl's width may vary and may be positioned centrally or offset on the associated female or male portion's surface.

The maximum external dimension of the male part and the maximum internal dimension of the female member (including the teeth and pawl) are very similar so as to provide a sliding fit of one in the other. This assures a tight fit (avoiding wobble due to poor tolerances) between the two members once connected. The bridging width (distance between two eyelets or limbs) of the two part device (tie or staple) can be varied once compression has been initiated, depending on the degree desired. The degree of compression may typically be a reduction of 2-5 millimetres of the device length, but may be more if desired or dependent on the staple size and the location being used.

The dimensions of the bone tie or staple implant will vary according to the application. In the human hand, or a small animal, the device may be 10mm long following compression. In large bones eg. femur, the device may be 30 mm long following compression. The wall thickness will vary according to the application and material used and may range from 1 mm to 3 mm or more.

In the embodiments of FIGS. 2-4 the body portion 16 of the sleeve member has a rectangular outer cross section, however it may have other outer cross sectional shapes, such as a rounded rectangular cross section.

The surface finish of the device is preferably polished or very smooth to allow engagement of the two members and gliding during compression. The limbs may be rectangular or square in cross section, also with a smooth surface. The limbs may have barbs or serrations on any surface, to prevent or limit pull out from bone. The technique to achieve a polished finish may be electro-polishing or any other known process.

The staple or tie may be made from medical grade stainless steel or titanium alloy, produced by any suitable manufacturing method, such as casting or additive manufacturing. The staple is packaged sterilised and single use. Each member is preferably of unitary construction (single piece, rather than an assembly). The devices may be sterilized and hermetically packages individually in matched counterpart pairs, or in groups.

The staples in accordance with the invention may be applied by the use of a staple holder is used to hold the two-part assembled staple together. This is a clamp with a ‘C’ shape (in cross section) part at one end, that fits around the periphery of the staple connection mechanism. It has ring and ratchet handles at the other end to allow the progressive application of compression at the staple end.

A staple impactor may be used to make the device flush with bone during insertion. This is a metal handle with a strikeable plate at one end for a hammer to hit. At the other end is a flat metal surface that can be placed over the staple. The staple engagement end is ‘C’ shape (in cross section) but shallow enough on its two sides to allow the staple to be completely flush with bone without the impactor contacting bone.

A compression tool may be used to compress the staple members together. This could be a non-disposable, reusable scissor device, which engages the edges of the two staple peripheries with the limbs flush in the bone. When the handles are bought together, it in turn compresses the staple to the desired amount. The compression tool fits either around or within the periphery of the staple. The connection may be helped by the provision of one or more notches or tabs in/on the staple.

For use in joint fusion surgery, the two bone surfaces are prepared in the usual way known to the skilled person. A drill hole is made into each bone, at a defined distance apart. The staple device is assembled by joining the male and female members but not compressed. Each limb of the staple is the same distance apart as the pilot holes.

The staple is held in a staple holder, allowing ease of insertion and preventing unintended compression (and so change in size) of the staple prior to implantation. The staple is inserted into the bone via the drill holes by hand utilising the staple holder. The staple bridge portion is made flush onto the bone surface, across the fusion site, using the staple impactor.

The compression tool is placed at the staple periphery (the male and female ends). The compression tool handles are squeezed together, so compressing (reducing the width) of the staple by the desired degree. This applies and maintains a compression force across the two bone surfaces to be fused.

Removal of the staple, if desired, is achieved by pulling out the complete construct (male and female parts) out if the drill holes. The staple cannot easily be disassembled and should be discarded.

The staple or bone tie connection mechanism may vary in length in any variation to allow the limbs to be offset. By replacing the limb of the staple with an eyelet and screw one, may increase the pull out strength from bone and the rigidity of the construct. The eyelet hole may be threaded to improve the stability and rigidity of the fixation where the screw head is also threaded at an upper end to engage with the eyelet hole, while the lower end of the screw is threaded for bone engagement.

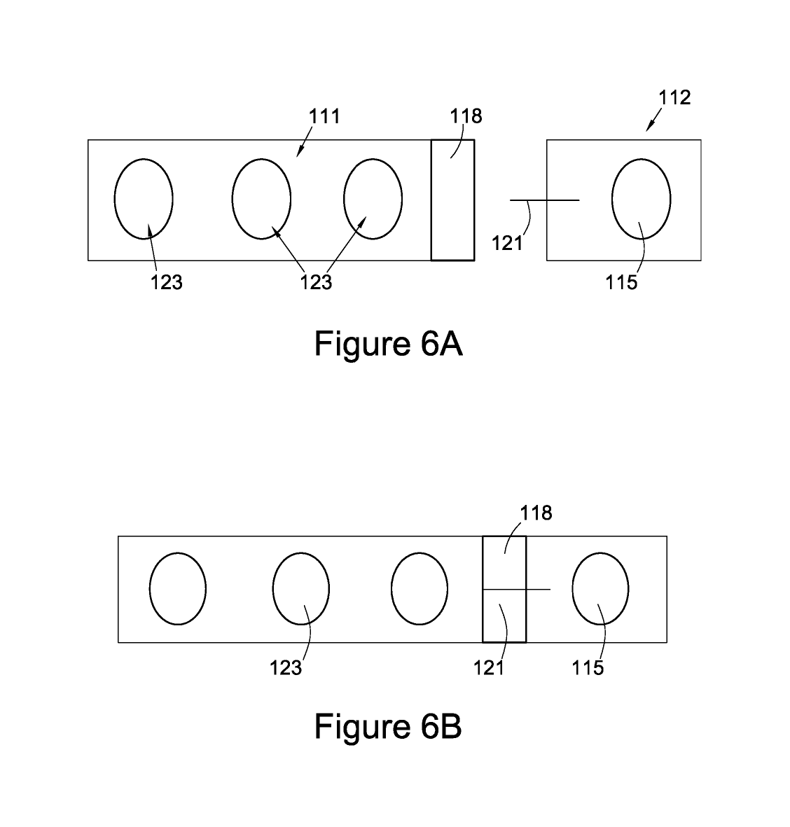

The bone tie may be in the form of a plate (FIG. 6A & B) rather than a tie rod (FIG. 5A) or staple. This will give further fixation options for a fusion site or fracture of bone and so allow compression. In FIG. 6, in the female member 111 is shown as a plate with three fixing holes 123 and the male member 112 is a plate with one fixing hole (or eyelet) 115. The number of holes in the plate on either plate is not of course limited.

The ratchet and pawl engagement is shown schematically in FIGS. 5 and 6 by the male regions 121 and female regions 118.

In certain embodiments the female member and/or male member may include at least a portion that is bendable to allow the member to be shaped during implantation to suit the underlying anatomy at the implantation site. In other words, the bone tie may be bendable at some point along its length. For example, the bendable portion may be manually shaped to suit the anatomy at the implantation site. The female member and/or male member may include a plate portion toward the distal end region which is bendable.

The bone tie may, rather than being straight, have some curvature. This could be curvature in any plane (e.g. coronal, sagittal, axial). In such embodiments, the bone tie could be manufactured with a predetermined curvature.

In further embodiments there may be provided bone ties or staples in which multiple devices are integrated with each other, so that two engagement portions, or three, or more, may be included in a single device.

1. A surgical bone tie for use in joining abutting bone surfaces for fusing or knitting of the bone together, the tie comprising (or consisting of) first and second pieces each having a proximal end region and a distal end region which define a longitudinal axis therebetween,
wherein the distal end regions are each provided with a mounting feature for fixing, or permitting the fixing, of each piece to respective underlying portions of bone, wherein the proximal end regions of each pieces are adapted for engagement together along said axis so that one proximal end region may be accommodated by the other proximal end region to provide a bridge between the respective distal end regions, the engagement permitting one way travel of one piece progressively towards the other so that the bridge length becomes progressively smaller until a desired amount of separation between the distal end regions is obtained, and a desired compression between the abutting bones is obtained,
wherein the engagement between the proximal end regions comprises an axially oriented internal ratchet and pawl connection, the first piece of the bone tie being an elongate female sleeve member with a hollow cross-section having a top surface facing away from the bone, an underside and two side surfaces and the second piece of the bone tie being a male member that forms a sliding fit in the female sleeve member, the female sleeve member having a pawl on its top surface, or one or two pawls on its side surfaces, the male member having corresponding ratchet teeth and the or each pawl comprising a cantilever spring member oriented along an axial direction, a free end of which is biased against the corresponding ratchet teeth on the male member for sequential engagement with said ratchet teeth.

2. A bone tie as claimed in claim 1 wherein one or both distal end regions are provided with an eyelet for receiving a bone screw or bolt.

3. A bone tie as claimed in claim 2 wherein both distal end regions are provided with an eyelet for receiving a fixing element such as a bone screw or bolt or spike.

4. A bone tie as claimed in claim 1 wherein one or both distal end regions are provided with a limb to be driven into the underlying bone for fixing thereto, which limb is preferably generally perpendicularly-oriented.

5. A bone tie as claimed in claim 4 wherein both distal end regions are provided with a generally perpendicularly-oriented limb to be driven into the underlying bone for fixing thereto.

6.-7. (canceled)

8. A bone tie as claimed in claim 1 wherein the cantilever spring member projects axially towards the proximal end region of the associated tie piece.

9. A bone tie as claimed in claim 1 wherein the cantilever spring member projects axially towards the distal end region of the associated tie piece.

10. (canceled)

11. A bone tie as claimed in claim 1 wherein the pawl and ratchet are integrally formed with each respective tie piece.

12.-13. (canceled)
